(19) United States
(12) Patent Application Publication
(10) Pub. No.: US 2024/0366433 A1
(43) Pub. Date: Nov. 7, 2024

(54) METHOD AND APPARATUS FOR PRODUCING ABSORBENT SANITARY ARTICLES AND RELATIVE ABSORBENT SANITARY ARTICLE

(71) Applicant: Fameccanica.Data S.p.A., San Giovanni Teatino Chieti (IT)

(72) Inventor: Gabriele SABLONE, San Giovanni Teatino Chieti (IT)

(21) Appl. No.: 18/770,047

(22) Filed: Jul. 11, 2024

Related U.S. Application Data
(62) Division of application No. 17/075,806, filed on Oct. 21, 2020, now Pat. No. 12,064,324.

(30) Foreign Application Priority Data
Oct. 25, 2019 (IT) 102019000019785

(51) Int. Cl. A61F 13/15; A61F 13/496

(52) U.S. Cl. CPC A61F 13/15658; A61F 13/15723; A61F 13/496

(57) ABSTRACT

A method and apparatus for producing absorbent sanitary articles according to a Machine Direction technique, each sanitary article having an absorbent body, a front waistband and a rear waistband having different lengths from each other.

### CROSS REFERENCE TO RELATED APPLICATIONS

This application is a divisional of U.S. application Ser. No. 17/075,806 filed on Oct. 21, 2020, and claims priority to Italian Patent Application No. 102019000019785 filed on Oct. 25, 2019, the entire disclosure of these applications is incorporated herein by reference.

### FIELD OF THE INVENTION

The present invention relates to a method and an apparatus for producing absorbent sanitary articles.

The invention was developed with particular regard to producing absorbent sanitary articles that can be worn as pants, for example, the so-called training pants. The invention is applicable both for producing permanently closed-type absorbent sanitary articles and those of the openable and reclosable-type.

The present invention relates, in particular, to a method and an apparatus for producing absorbent sanitary articles according to the so-called “Machine Direction” production technique.

### DESCRIPTION OF THE PRIOR ART

Absorbent sanitary articles known as “training pants” are normally formed by an absorbent body having an elongated shape along a longitudinal axis and two transverse waistbands (a front waistband and a rear waistband) applied to opposite ends of the absorbent body on the surface that, during use, faces towards the clothing. The transverse waistbands may be elasticized and can be attached to each other permanently or by opening and reclosable closing formations.

For producing absorbent sanitary articles composed of an absorbent body of elongated shape and two transverse waistbands, a production technique called “Cross Direction” is normally used, which consists in forming two continuous parallel sheets, forming the front and rear waistbands, which advance in a machine direction and applying the absorbent bodies between the continuous sheets. The absorbent bodies are oriented transversely with respect to the machine direction and are attached at their opposite ends to the continuous sheets forming the waistbands.

It is known that using this production technique the absorbent bodies are not always applied in a precise way and protrude with respect to the waistbands. In the latter case, a cut is made to eliminate the protruding portion, which involves scraps and requires appropriate cutting devices equipped with scrap-removal units.

In the case that absorbent bodies are applied to the continuous sheets by gluing, it is known that glue is present on the protruding portion of the absorbent bodies to be removed. The presence of glue on the part to be removed further complicates the cutting and removal of the scrap. Examples of methods for producing absorbent sanitary articles according to the Cross Direction technique are described in documents EP-A-1013251, IT1379452, IT1410464 and IT1410465 by the same Applicant.

The Cross Direction production technique makes it possible to produce absorbent sanitary articles wherein the absorbent body is shorter than the overall length of the finished product, i.e. with the outer edges of the waistbands protruding beyond the transverse edges of the absorbent bodies.

However, with the Cross Direction production technique, it would be very complex to produce absorbent sanitary articles wherein the two waistbands have different lengths, for example, with the rear waistband having a longer length than the front waistband, so that the closing formations are placed on the front of the user instead of on the sides. In the case wherein it is required to produce absorbent sanitary articles with front and rear waistbands with different lengths with the Cross Directions production technique, it would be necessary to use very complex equipment with a high production of scraps.

### OBJECT AND SUMMARY OF THE INVENTION

The present invention aims to provide a method and apparatus for producing absorbent sanitary articles that overcome the problems of the prior art.

In particular, the present invention aims to provide a method and an apparatus that allow the production of absorbent sanitary articles without scraps, composed of an absorbent body with an elongated shape, and two waistbands with different lengths.

According to the present invention, this object is achieved by a method having the characteristics forming the subject of claims 1 and 5.

According to another aspect, the present invention relates to an absorbent sanitary article having the characteristics forming the subject of claim 8.

The claims form an integral part of the disclosure provided here in relation to the invention.

### BRIEF DESCRIPTION OF THE DRAWINGS

The present invention will now be described in detail with reference to the attached drawings, given purely by way of non-limiting example, wherein:

FIG. 1 is a plan schematic view of an embodiment of an absorbent sanitary article according to the present invention;

FIG. 2 is a schematic side view of an embodiment of an apparatus configured for producing absorbent sanitary articles shaped as illustrated in FIG. 1; and

FIGS. 3-9 are plan views illustrating the steps of a method for producing absorbent sanitary articles corresponding to the cross-sections of the apparatus indicated by arrows III-IX of FIG. 2.

It will be appreciated that the various figures may not be represented on the same scale.

### DETAILED DESCRIPTION

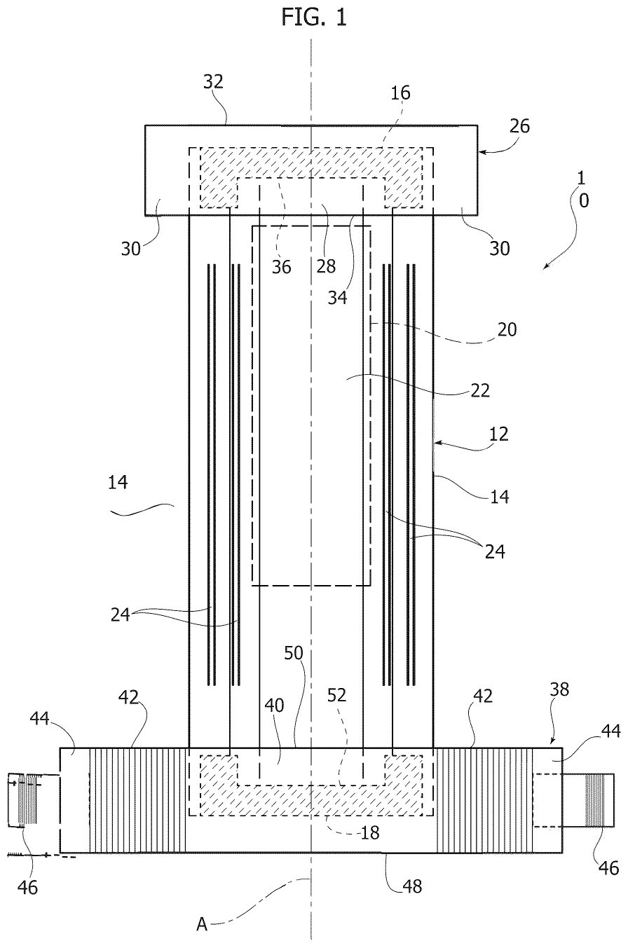

With reference to FIG. 1, numeral 10 indicates an absorbent sanitary article illustrated in an open and extended position. The absorbent sanitary article 10 illustrated—by way of example—in FIG. 1 is an openable and reclosable training pant. However, it is understood that the present invention may also apply to absorbent sanitary articles of another type, for example, incontinence pads for adults either permanently closed or not.

The absorbent sanitary article 10 comprises an absorbent body 12 having an elongated shape along a longitudinal axis A. The absorbent body 12 has two longitudinal sides 14 that extend along a direction defined by the longitudinal axis A. The longitudinal sides 14 may be parallel to the longitudinal axis A. The absorbent body 12 has a front transverse side 16 and a rear transverse side 18 transverse to the longitudinal axis A. The front transverse side 16 and/or the rear transverse side 18 may be perpendicular to the longitudinal axis A.

The absorbent body 12 comprises an absorbent core 20 enclosed between an inner sheet of liquid-permeable material (topsheet) and an outer sheet of impermeable material (backsheet). For example, the absorbent core 20 may be sandwiched between the inner sheet and the outer sheet. Additional absorbent layers may be comprised between the inner sheet and the outer sheet of the absorbent core 20 such as, for example, an Acquisition Diffusion Layer, commonly referred to as ADL. The topsheet has a surface intended to face, during use, towards the user's body. This topsheet surface is indicated in the drawings with the reference 22 and refers to the inner surface of the absorbent body 12.

The absorbent body 12 may comprise additional components as is customary in the field, such as, for example, elastic elements for the legs 24 (leg cuffs) applied on the inner surface 22 and extending parallel to the longitudinal axis A, and/or diffusion layers of the liquids, etc.

The absorbent sanitary article 10 comprises a front waistband 26 having an elongated shape in a transverse direction. In the embodiment illustrated in the figures, the front waistband 26 may be formed by a non-elastic sheet, for example, a sheet of non-woven fabric. The front waistband 26 has a central portion 28 attached to the inner surface 22 of the absorbent body 12, and two side portions 30 which extend in a transverse direction outwardly with respect to the longitudinal sides 14 of the absorbent body 12. The front waistband 26 has an outer transverse edge 32 and an inner transverse edge 34. Along the longitudinal axis A, the outer transverse edge 32 extends outwardly with respect to the front transverse side 16 of the absorbent body 12.

In a possible embodiment, the front waistband 26 may be attached to the inner surface 22 of the absorbent body 12 along a front attachment area 36 having a “C” shape facing the center of the absorbent body 12. The attachment area 36 may be formed of a layer of adhesive. In the example illustrated in the Figures, the front attachment area 36 has a central zone with the shape of an elongated rectangle in the transverse direction, and adjacent to the front transverse side 16 of the absorbent body 12, and two side areas having the shape of elongated rectangles in the longitudinal direction, and adjacent to the longitudinal sides 14 of the absorbent body 12. This arrangement of the attachment area 36 allows a pocket to be formed between the central portion 28 of the front waistband 26 and the inner surface 22 of the absorbent body 12, open along the inner transverse edge 34 of the front waistband 26. This pocket, during use, may be useful for containing feces.

The absorbent sanitary article 10 comprises a rear waistband 38 having an elongated shape in a transverse direction. The rear waistband 38 has a central portion 40 attached to the inner surface 22 of the absorbent body 12, and two side portions 42 which protrude in a transverse direction outwardly with respect to the longitudinal sides 14 of the absorbent body 12.

The rear waistband 38 has a greater dimension in the transverse direction than the dimension in the transverse direction of the front waistband 26.

In a possible embodiment, the central portion 40 of the rear waistband 38 is not elasticized, and the side portions 42 are at least partly elasticized and elastically extendable in the transverse direction. The side portions 42 may have non-elasticized end regions 44 on which openable and reclosable closing formations 46 can be attached, which are configured to cooperate with the outer surfaces of the side portions 30 of the front waistband 26.

The rear waistband 38 has an outer transverse edge 48 and an inner transverse edge 50. Along the longitudinal axis A, the outer transverse edge 48 extends outwardly with respect to the rear transverse side 18 of the absorbent body 12.

In a possible embodiment, the central portion 40 of the rear waistband 38 is attached to the inner surface 32 of the absorbent body 12 along a rear attachment area 52 having a “C” shape facing the center of the absorbent body 12. The rear attachment area 52 may have a specular shape with respect to the front attachment area 36. The rear attachment area 52, may also have a central zone with the shape of an elongated rectangle in the transverse direction and adjacent to the rear transverse side 18 of the absorbent body 12, and two side areas having the shape of elongated rectangles in the longitudinal direction and adjacent to the longitudinal sides 14 of the absorbent body 12. In this case as well, a pocket may be formed between the central portion 40 of the rear waistband 38 and the inner surface 22 of the absorbent body 12, open along the inner transverse edge 50 of the rear waistband 38. The rear pocket may also be useful, in use, for collecting feces.

The absorbent sanitary article 10 has an overall length in the direction of the longitudinal axis A greater than the length of the absorbent body 12. The front and rear waistbands 26, 38 are applied to the inner surface of the absorbent body 12 over the transverse sides 16, 18. This avoids direct contact of the transverse edges 16, 18 of the absorbent body 12 with the user's skin, which could cause irritation.

The front and rear waistbands 26, 38 have different lengths and can be made of different materials. For example, in the illustrated embodiment, the front waistband 26 may have a transverse dimension smaller than the transverse dimension of the rear waistband 38, so that closing the absorbent sanitary article 10 can be carried out on the front of the user instead of on the sides.

In a possible embodiment, the absorbent body 12, the front waistband 26 and the rear waistband 38 may have rectangular shapes.

According to possible embodiments, the absorbent sanitary article 10 may comprise a connecting panel (frontal tape) configured to couple with the opening and closable closing formations 46. The connecting panel is provided with a connecting surface configured to couple with the openable and reclosable closing formations 46, and an attachment surface opposite the connecting surface attached to the front waistband 26 and the surface of the back sheet.

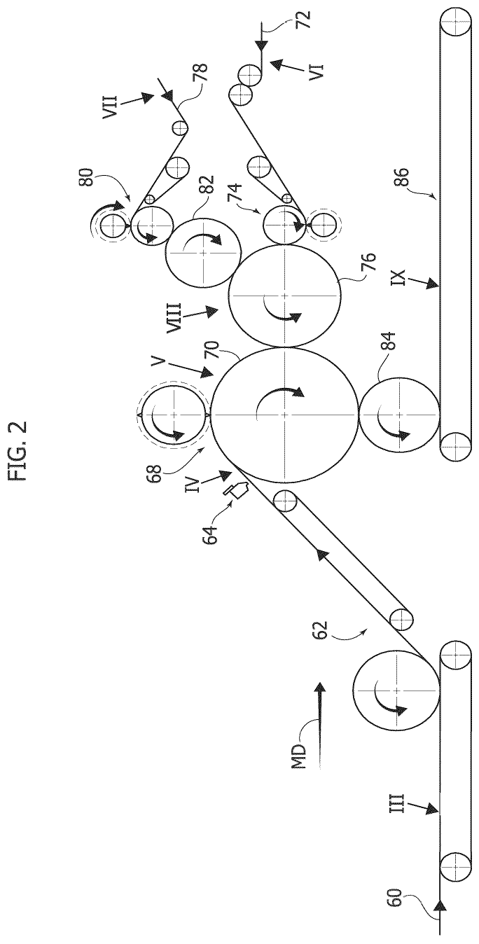

With reference to FIGS. 2-9, a possible embodiment of a method and an apparatus for producing an absorbent sanitary article 10 of the type illustrated in FIG. 1 will now be described.

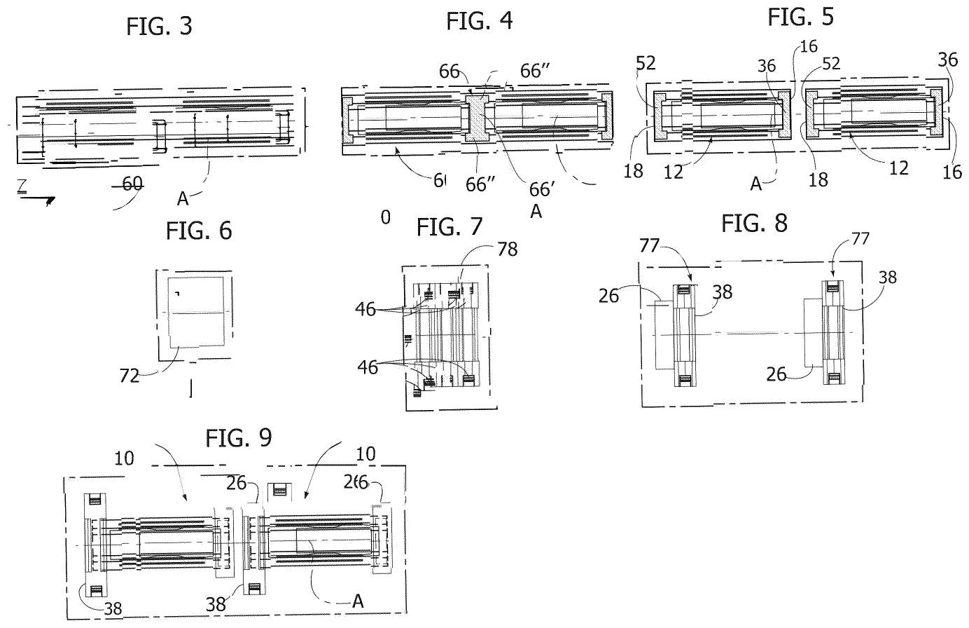

The method initially envisages the provision of a continuous absorbent composite web 60 (FIG. 3) having a longitudinal axis A and formed by a continuous succession of blanks of absorbent bodies. The continuous absorbent composite web 60 comprises an inner sheet of liquid-permeable material and an outer sheet of impermeable material, between which a continuous succession of absorbent cores 20 are enclosed, arranged with their respective longitudinal axes aligned with the longitudinal axis A, and spaced apart from each other in the longitudinal direction.

The continuous composite web 60 is produced according to the Machine Direction technique, in a manner well known in the sector of producing absorbent articles.

Referring to FIG. 2, the continuous absorbent composite web 60 is advanced along a machine direction MD along a transport unit 62.

A glue application unit 64 applies glue areas 66 (FIG. 4) on the inner surface of the absorbent continuous composite web 60, said areas being spaced apart in the direction of the longitudinal axis A. Each glue area 66 has an “I” shape, with a central zone 66′ elongated in the transverse direction and two side areas 66″ wider than the central zone.

After applying the glue, the continuous absorbent composite web 60 is cut along a transverse direction perpendicular to the longitudinal axis A in a cutting unit 68. At the outlet of the cutting unit 68, the continuous absorbent composite web 60 is divided into a continuous succession of absorbent bodies 12 (FIG. 5). The transverse cut is made through the glue areas 66, so that each absorbent body 12 has two C-shaped glue areas at its opposite ends, facing the center of the absorbent body (FIG. 5).

With reference to FIG. 2, after the transverse cut, the absorbent bodies 12 are retained on the outer surface of an anvil roller 70.

With reference to FIGS. 2 and 6, the method envisages forming a plurality of front waistbands starting from a continuous sheet 72, by means of transverse cuts made in a cut-and-slip unit 74. The front waistbands 26 at the outlet of the cut-and-slip unit 74 are held on a transfer wheel 76.

With reference to FIGS. 2 and 6, the method envisages forming a plurality of rear waistbands starting from a continuous web 78 by means of transverse cuts made in a cutting unit 80. The rear waistbands 38 at the outlet of the cutting unit 80 are applied onto the transfer wheel 76 by means of a repitch device 82.

With reference to FIG. 8, on the transfer wheel 76 downstream of the repitch device 82 there are pairs of waistbands spaced apart by a distance equal to the length of the absorbent sanitary articles 10. Each pair of waistbands 77 is formed by a front waistband 26 and a rear waistband 38 adjacent to each other.

The front and rear waistbands 26, 38 arranged on the transfer wheel 76—as indicated in FIG. 8—are applied to the ends of the absorbent bodies 12 that advance on the anvil roller 70.

The front and rear waistbands 26, 38 located on the transfer wheel 76 are in phase with the absorbent bodies 12, so that a front waistband 26 is applied to each absorbent body 12 at the front transverse side 16 and a rear waistband 38 at the rear transverse side 18.

The transfer wheel 76 sequentially applies the front waistband 26 of each pair of waistbands 77 onto the inner surface 22 of a first absorbent body 12, and the rear waistband 38 of the same pair of waistbands 77 on the inner surface 22 of a second absorbent body 12 following the first absorbent body 12.

The front and rear waistbands 26, 38 are applied to the glue areas of the absorbent bodies 12 and are attached to the absorbent bodies 12 by gluing.

In a possible alternative embodiment, the front waistbands 26 and the rear waistbands 38 may be welded to the inner surface 22 of the absorbent bodies 12. The welding may be performed by ultrasonic welding or thermowelding by means of a welding unit (not shown), which can cooperate with the anvil roller 70 and is located between the transfer wheel 76 and the detachment wheel 84. In this variant, the application of glue is not envisaged.

After applying the front and rear waistbands 26, 38 onto the absorbent bodies 12, the finished absorbent sanitary articles 10 are obtained. A detachment wheel 84 detaches the absorbent sanitary articles 10 from the anvil roller 70, and transfers them to an outlet conveyor 86. FIG. 9 shows the finished absorbent sanitary articles 10 in an extended position as they advance over the outlet conveyor 86.

Thereafter, the absorbent sanitary articles 10 can be folded around a central transverse axis and the side portions of the rear waistbands can be folded around longitudinal axes to bring the closing formations 46 into contact with the side portions of the front waistbands 26.

Of course, without prejudice to the principle of the invention, the details of construction and the embodiments can be widely varied with respect to those described and illustrated, without thereby departing from the scope of the invention as defined by the claims that follow.

1. An absorbent sanitary article, comprising:
an absorbent body having a shape elongated along a longitudinal axis, two longitudinal sides, a front transverse side and a rear transverse side perpendicular to said longitudinal axis, wherein the absorbent body has an inner surface of liquid-permeable material adapted to be placed in contact with a user's skin,
a front waistband having an elongated shape in a direction transverse to said longitudinal axis, wherein the front waistband has a central portion attached to said inner surface of said absorbent body and an outer transverse edge that extends outwardly with respect to said front transverse side along said longitudinal axis, and
a rear waistband having an elongated shape in said transverse direction, wherein said rear waistband has a central portion attached to said inner surface of said absorbent body and has a greater length in said transverse direction than a length in the transverse direction of the front waistband, and wherein the rear waistband has an outer transverse edge which extends outwardly with respect to said rear transverse side along said longitudinal axis.

2. The article according to claim 1, wherein the central portion of the front waistband is attached to the central body along a front attaching area having a “C” shape facing towards a center of the absorbent article, so that a front pocket is formed between the front waistband and the inner surface of the absorbent body, open along an inner transverse edge of the front waistband.

3. The article according to claim 2, wherein the central portion of the rear waistband is attached to the absorbent body along a rear attaching area having a “C” shape facing towards the center of the absorbent article, so that a rear pocket is formed between the rear waistband and the inner surface of the absorbent body, open along an inner transverse edge of the rear waistband.

4. The article according to claim 1, wherein the front waistband is formed of and comprises a first material, and wherein the rear waistband is formed of and comprises a second material that is different than the first material.
